(19) United States
(12) Patent Application Publication
(10) Pub. No.: US 2021/0309649 A1
(43) Pub. Date: Oct. 7, 2021

(54) SUBSTITUTED GLUTARIMIDE DERIVATIVES

(71) Applicant: BASF SE, Ludwigshafen (DE)

(72) Inventors: Nikolas Huwyler, Ludwigshafen (DE); Karsten Koerber, Ludwigshafen (DE); Arun Narine, Ludwigshafen (DE)

(21) Appl. No.: 17/264,979

(22) PCT Filed: Aug. 15, 2019
(86) PCT No.: PCT/EP2019/071916

§ 371 Date: Feb. 1, 2021

(30) Foreign Application Priority Data
Aug. 22, 2018 (EP) 18190125.7

(51) Int. Cl. C07D 413/12; A01N 43/80; A01N 43/40; C07D 401/12; C07D 211/98

(52) U.S. Cl. CPC C07D 413/12; A01N 43/80; C07D 211/98; C07D 401/12; A01N 43/40

(57) ABSTRACT

The invention relates to glutarimide compounds of formula I, (I) wherein the variables have the meanings as defined in the specification, to compositions comprising them, to active compound combinations comprising them, and to their use for protecting growing plants and animals from attack or infestation by invertebrate pests, furthermore, to seed comprising such compounds.

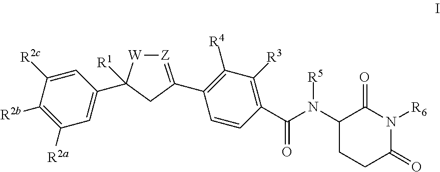

The invention relates to glutarimide compounds of formula I

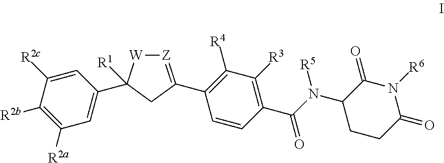

wherein
W—Z is —O—N═, —CH₂—N═, or —CH₂—CH═;
R¹ halomethyl;
R^{2a} halogen, halomethyl, or halomethoxy;
R^{2b}, R^{2c} are independently H, or as defined for R^{2a};
R³ is halogen, CN, NO2, C₁-C₂-alkyl, halomethyl, C₁-C₂-alkoxy, S(O)ₘ—C₁-C₂-alkyl, C₁-C₂-haloalkoxy, or S(O)ₘ—C₁-C₂-haloalkyl;
R⁴ is H, or as defined for R³; or
R³ and R⁴ form together with the C-atoms they are bound to a 5-, or 6-membered saturated, partially, or fully unsaturated carbocyclic ring;
R⁵, R⁶ are independently H, CN, C₁-C₁₀-alkyl, C₃-C₈-cycloalkyl, C₂-C₁₀-alkenyl, C₃-C₈-cycloalkenyl, C₂-C₁₀-alkynyl, OR¹⁰, S(O)ₘR¹⁰, S(O)ₘN(R¹⁰)₂, N(R¹⁰)₂, which aliphatic groups are unsubstituted, partially or fully halogenated and/or substituted with one or more R^{a}; phenyl which is unsubstituted or substituted with one or more R^{A}; and 3- to 7-membered saturated, partially or fully unsaturated heterocycle comprising 1, 2 or 3 heteroatoms 0, N(O)ₙ or S(O)ₘ as ring members, which heterocycle is unsubstituted or substituted with one or more R^{A},
   R¹⁰ is independently H, C₁-C₆-alkyl, C₁-C₆-haloalkyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted or substituted with one or more R^{a},
   R^{a} is CN, N₃, NO₂, SCN, SF₅, Si(C₁-C₄-alkyl)₃, OR^{a1}, OSO₂R^{a1}, S(O)ₘR^{a1}, N(R^{a2})R^{a3}, C(═O)N(R^{a2})R^{a3}, C(═S)N(R^{a2})R^{a3}, C(═O)R^{a1}, C(═O)OR^{a1}, CH═NOR^{a1}, C₃-C₈-cycloalkyl, C₃-C₈-halocycloalkyl, which cyclic moieties may be substituted with Rao; phenyl which is unsubstituted or substituted with one or more R^{A}; and 3- to 7-membered saturated, partially or fully unsaturated heterocycle comprising 1, 2 or 3 heteroatoms 0, N(O)ₙ or S(O)ₘ as ring members, which heterocycle is unsubstituted or substituted with one or more R^{A},
   m is 0, 1, or 2;
   n is 0, or 1;
      R^{a1} H, C₁-C₆-alkyl, C₁-C₆-haloalkyl, C₂-C₄-alkynyl, CH₂—CN, C₃-C₆-cycloalkyl, C₃-C₆-halocycloalkyl, C₃-C₆-cycloalkylmethyl, C₃-C₆-halocycloalkylmethyl, phenyl and hetaryl which aromatic rings are unsubstituted or partially or fully substituted with R^{A};
      R^{a2} is H, or C₁-C₆-alkyl,
      R^{a3} is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, or C₃-C₆-cycloalkyl, C₃-C₆-halocycloalkyl, C₃-C₆-cycloalkylmethyl, or C₃-C₆-halocycloalkylmethyl which rings are unsubstituted or substituted with a cyano;
      R^{a4} is independently OH, CN, C₁-C₆-alkoxy, C₁-C₆-haloalkoxy, S(O)ₘ—C₁-C₆-alkyl, S(O)ₘ—C₁-C₆-haloalkyl, C(═O)N(R^{a2})R^{a3}, C₃-C₆-cycloalkyl, or C₃-C₆-halocycloalkyl which cycles are unsubstituted or substituted with one or more R^{a11}; or phenyl, partially or fully unsaturated heterocycle which rings are unsubstituted or substituted with one or more R^{A};
      R^{a11} is independently OH, cyano, C₁-C₂-alkyl, or C₁-C₂-haloalkyl;
      R^{A} is independently selected from halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, S(O)ₘ—C₁-C₄-alkyl, S(O)ₘ—C₁-C₄-haloalkyl, C₁-C₄-alkylcarbonyl, C₁-C₄-haloalkylcarbonyl, C(═O)N(R^{a2})R^{a3}; or
      two R^{A} present on the same carbon atom of a saturated or partially saturated ring may form together ═O or ═S; or
      two R^{A} present on the same S or SO ring member of a heterocyclic ring may together form a group ═N(C₁-C₆-alkyl), ═NO(C₁-C₆-alkyl), ═NN(H)(C₁-C₆-alkyl) or ═NN(C₁-C₆-alkyl)₂;
         and the N-oxides, stereoisomers and agriculturally or veterinarily acceptable salts thereof.

The invention also provides an agricultural composition comprising at least one compound of formula I, a stereoisomer thereof and/or an agriculturally acceptable salt thereof and at least one liquid and/or solid carrier, especially at least one inert liquid and/or solid agriculturally acceptable carrier.

The invention also provides a veterinary composition comprising at least one compound of formula I, a stereoisomer thereof and/or a veterinarily acceptable salt thereof and at least one liquid and/or solid carrier, especially at least one inert veterinarily liquid and/or solid acceptable carrier.

The invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The invention also relates to plant propagation material, in particular seed, comprising at least one compound of formula I and/or an agriculturally acceptable salt thereof.

The invention further relates to a method for treating or protecting an animal from infestation or infection by parasites which comprises bringing the animal in contact with a parasitically effective amount of a compound of formula I or a veterinarily acceptable salt thereof. Bringing the animal in contact with the compound I, its salt or the veterinary composition of the invention means applying or administering it to the animal.

JP 2007/091708, WO 2007/123853, WO 2007/123855, WO 2008/128711, WO 2010/020522, WO 2013/037626, WO 2017/050921, and WO 2017/050922 describe structurally closely related active compounds. These compounds are mentioned to be useful for combating invertebrate pests.

Nevertheless, there remains a need for highly effective and versatile agents for combating invertebrate pests. It is therefore an object of the invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

Compounds of formula I can be prepared by reacting an activated carboxylic acid derivative of formula II or the corresponding carboxylic acid IIa with a compound of formula III in an amidation reaction. X in formula II denotes a leaving group, preferably halogen such as e.g. Cl or Br, or C₁-C₆-alkoxy such as OCH₃ or OC₂H₅. Aminoglutarimide III is preferably used as its ammonium salt, wherein Y is an anion, preferably a halogenide such as Cl or Br.

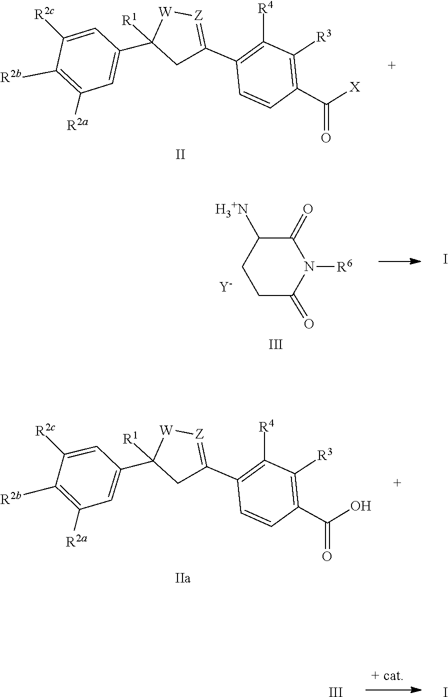

The amidation reaction is usually carried out with the acid chlorides or by prior transformation of carboxylic acids of formula IIa with oxalyl chloride [(COCl)₂] or thionylchloride (SOCl₂) to the corresponding acid chlorides, followed by reaction with the amine of formula III. Suitable reaction conditions are described in literature, e.g. in WO 2004/22536. The reaction is generally carried out in the presence of an organic base such as triethylamine (Et₃N), N,N-diisopropylethylamine (iPr₂NEt), pyridine, substituted pyridines such as collidine or lutidine, or the like. Optionally a nucleophilic catalyst such as 4-(N,N-dimethylamino)pyridine (“DMAP”) can be employed in the reaction. Suitable solvents are halogenated hydrocarbons such as, dichloromethane, chloroform, and chlorobenzene, or polar aprotic solvents such as THF, and N,N-dimethylformamide (DMF), or aromatic hydrocarbons such as benzene, toluene, o-, m-, and p-xylene, or mixtures thereof. The transformation is usually carried out at temperatures from −40° C. to 100° C., preferably from 0° C. to 30° C. The starting materials are generally reacted with one another in equimolar amounts. In terms of yield, it may be advantageous to employ an excess of III, based on II.

Alternatively, amidation of the carboxylic acid IIa is carried out in the presence of a coupling reagent. Suitable coupling reagents (activators) are known and are, e.g. selected from carbodiimides, such as N,N-dicyclohexylcarbodiimide (“DCC”) and N,N-diisopropylcarbodiimide (“DCI”), benzotriazole derivatives such as 1-[bis(dimethylamino)methylene]-1H-1,2,3-triazo-lo[4,5-b]pyridinium 3-oxid hexafluorophosphate (“HATU”), O-(benzotriazol-1-yl)-N,N,N′,N′-tetra-methyluronium hexafluorophosphate (“HBTU”), and 1-[bis(dimethylamino)methylen]-5-chloro-benzotriazolium 3-oxide hexafluorophosphate (“HCTU”), or phosphonium-derived activators, such as (Benzotriazol-1-yloxy)tris(dimethylamino)phosphonium hexafluorophosphate (“BOP”), (benzotriazol-1-yl-oxytripyrrolidinophosphonium hexafluorophosphate) (“Py-BOP”), bromotripyrrolidinophosphonium hexafluorophosphate (“Py-BrOP”). Generally, the activator is used in excess. The benzotriazole and phosphonium coupling reagents are generally used in a basic medium. Preferably, bromotripyrrolidinophosphonium hexafluorophosphate (“Py-BrOP”) is used as the coupling reagent (activator). Suitable reaction conditions are described in the literature, e.g. in WO2015/128358. The reaction is generally carried out in the presence of a base such as a tertiary amine base like iPr₂NEt, Et₃N. Suitable solvents are, e.g., halogenated hydrocarbons such as dichloromethane, chloroform, and chlorobenzene. The transformation is usually carried out at temperatures from 0° C. to 100° C., preferably from 10° C. to 40° C. The starting materials are generally reacted with one another in equimolar amounts. In terms of yield, it may be advantageous to employ an excess of III, based on II.

Introduction of group R⁵ being different from hydrogen is preferably made to formula III compounds. Alternatively, such R⁵ can also be introduced to formula VI, or to formula I compounds wherein R⁵ is H.

Compounds of formula III can be obtained by a reaction sequence starting with the mono-amidation of a glutamic acid derivative of formula IV, wherein PG is a protective group, such as benzyloxycarbonyl (“Cbz”), benzyl (“Bn”), tert-butylcarbonyl (“Boc”), or acetyl (“Ac”). Compounds of formula IV are commercially available, or can be made by standard methods of organic chemistry known to a person skilled in the art.

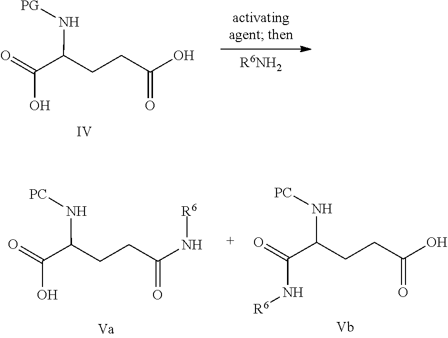

Suitable reaction conditions for the preparation of compounds of formula Va and Vb by mono-amidation of a compound of formula IV can de found in the literature, for example in A. Asati et al., Angew. Chem. Int. Ed. 2009, 48, 2308-2312. The reaction is generally carried out by treating a glutamic acid derivative of formula IV with a carboxylic acid activator such as, e.g., thionyl chloride (SOCl₂), 1,1′-carbonyldiimidazole, or carbodiimides, such as N,N-dicyclohexylcarbodiimide (“DCC”) and N,N-diisopropylcarbodiimide (“DCI”), or the like, followed, after the appropriate time, by the addition of the amine of formula R⁶NH₂. Suitable solvents are, e.g., halogenated hydrocarbons such as, dichloromethane, chloroform, and chlorobenzene, or polar aprotic solvents such as THF, N,N-dimethylformamide (DMF), and dimethyl sulfoxide (DMSO), or mixtures thereof. Optionally, the reaction can be carried out in the presence of an additional organic base such as, NEt₃, N-ethyl-N,N-diisopropylamine (iPr₂NEt), pyridine, or substituted pyridines such as collidine or lutidine, and the like. The transformation is usually carried out at temperatures from −50° C. to 50° C., preferably from 0° C. to 25° C. The starting materials are generally reacted with one another in equimolar amounts. In terms of yield, it may be advantageous to employ a slight excess of the carboxylic acid activator and R⁶NH₂, based on IV. From the mono-amidated compounds of formula Va and/or Vb, the desired compounds of formula III can be accessed by a cyclization reaction.

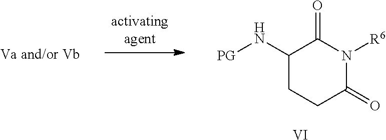

Suitable reaction conditions for the cyclization of compounds of formula Va and Vb can de found in the literature, for example in S. G. Davies et al., J. Chem. Soc., Perkin Trans. 1, 1998, 2635-2643. The reaction is generally carried out in the presence of a carboxylic acid activator such as, e.g., thionyl chloride (SOCl₂), 1,1′-carbonyldiimidazole, or carbodiimides, such as N,N-dicyclohexylcarbodiimide (“DCC”) and N,N-diisopropylcarbodiimide (“DCI”), or the like and performed at temperatures from 0° C. to 100° C., preferably from 25° C. to 75° C. Suitable solvents are, e.g., halogenated hydrocarbons such as, dichloromethane, chloroform, and chlorobenzene, or polar aprotic solvents such as THF, N,N-dimethylformamide (DMF), and dimethyl sulfoxide (DMSO), or mixtures thereof. Optionally, the reaction can be carried out in the presence of an additional organic base such as, NEt₃, N-ethyl-N,N-diisopropylamine (iPr₂NEt), pyridine, or substituted pyridines such as collidine or lutidine, and the like. If desired, the mono-amidation and cyclization step can be carried out in one pot without intermediate isolation of Va and Vb.

Elimination of the protecting group can be effected under conditions generally known in the art, e.g. benzyloxycarbonyl (“Cbz”) is cleaved under acidic conditions, using a mineral acid such as, for example, HBr in acetic acid [cf. T. Polonski, J. Chem. Soc. Perkin Trans. 11988, 629-637]. Alternatively, the benzyloxycarbonyl (“Cbz”) can be cleaved under hydrogenolytic conditions in the presence of a H₂ atmosphere, and a palladium catalyst such as, e.g., palladium on carbon (“Pd/C”) [cf. H. Iding et al., Tetrahedron 2004, 647-653], Pd(OH)₂ on carbon (“Pearlman cata lyst”) [cf. J. Maddaluno et al. Tetrahedron: Asymmetry 1992, 3, 1239-1242].

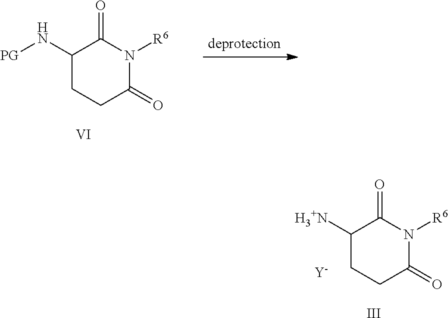

The starting materials required for preparing the compounds I are commercially available, or known from literature [cf. WO2014/029639; WO2010/72781] or can be prepared in accordance with the literature cited. Starting synthesis of compounds IV from commercially availably R-as-partic acid and following the above given synthesis yields in R-configured compounds III, and compounds I with R configuration in the glutarimid group, which correspond to formula I.a:

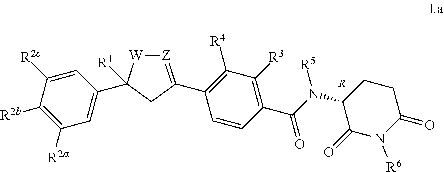

As a rule, the compounds of formula I including their stereoisomers, salts, and N-oxides, and their precursors in the synthesis process, can be prepared by the methods described above. If individual compounds cannot be prepared via the above-described routes, they can be prepared by derivatization of other compounds I or the respective precursor or by customary modifications of the synthesis routes described. For example, in individual cases, certain compounds of formula I can advantageously be prepared from other compounds of formula I by derivatization, e.g. by ester hydrolysis, amidation, esterification, ether cleavage, olefination, reduction, oxidation and the like, or by customary modifications of the synthesis routes described.

The reaction mixtures are worked up in the customary manner, e.g. by mixing with water, separating the phases, and, if appropriate, purifying the crude products by chromatography, e.g. on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

However, if the synthesis yields mixtures of isomers, a separation is generally not necessarily required since in some cases the individual isomers can be interconverted during work-up for use or during application (e.g. under the action of light, acids or bases). Such conversions may also take place after use, e.g. in the treatment of plants in the treated plant.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (Me), ethyl (Et), n-propyl, iso-propyl, n-butyl, 2-butyl, iso-butyl (iBu), tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoro-methoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2dichloro-2-fluorethoxy, 2,2,2-trichloroethoxy, penta-fluoroethoxy and the like.

The term “alkylthio “(alkylsulfanyl: S-alkyl)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom. The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: S(═O)—C₁-C₆-alkyl), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (S(═O)₂-alkyl) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the carbon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pen-tyn-1-yl, 3-pentyn-1-yl, 4-pentyn-1-yl, 1-methylbut-2-yn-1-yl, 1-ethylprop-2-yn-1-yl and the like. The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloal-kylthio denotes in each case a monocyclic cycloaliphatic radical having usually from 3 to 10 or from 3 to 6 carbon atoms, such as cyclopropyl (cPr), cyclobutyl, cyclopentyl, cyclohexyl, cyclo-heptyl, cyclooctyl, cyclononyl, and cyclodecyl, or cyclopropyl (c-C₃H₅), cyclobutyl (c-C₄H₇), cyclopentyl (c-C₅H₉), and cyclohexyl (c-C₆H₁₁).

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-fluo-rocyclopropyl, 1,2-, 2,2- and 2,3-difluorocyclopropyl, 1,2,2-trifluorocyclopropyl, 2,2,3,3-tetrafluo-rocyclpropyl, 1- and 2-chlorocyclopropyl, 1,2-, 2,2- and 2,3-dichlorocyclopropyl, 1,2,2-trichloro-cyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlo-rocyclopentyl, and the like.

The term “cycloalkenyl” as used herein and in the cycloalkenyl moieties of cycloalkenyloxy and cycloalkenylthio denotes in each case a monocyclic singly unsaturated non-aromatic radical having usually from 3 to 10, e.g. 3 or 4 or from 5 to 10 carbon atoms, preferably from 3- to 8 carbon atoms. Exemplary cycloalkenyl groups include cyclopropenyl, cycloheptenyl or cycloocte-nyl.

The term “halocycloalkenyl” as used herein and in the halocycloalkenyl moieties of halocyclo-alkenyloxy and halocycloalkenylthio denotes in each case a monocyclic singly unsaturated non-aromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3-difluorocyclopropen-1-yl and 3,3-dichlorocyclopropen-1-yl.

The term “cycloalkenylalkyl” refers to a cycloalkenyl group as defined above which is bonded via an alkylene group, such as a C₁-C₅-alkyl group or a C₁-C₄-alkyl group, in particular a methylene group (=cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered monocyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 5- or 6-membered, in particular 6-membered monocyclic heterocyclic non-aromatic radicals. The heterocyclic non-aromatic radicals usually comprise 1, 2 or 3 heteroatoms selected from N, O and S as ring members, wherein S-atoms as ring members may be present as S, SO or SO₂. Examples of 5- or 6-membered heterocyclic radicals comprise saturated or unsaturated, non-aromatic heterocyclic rings, such as 2- and 3-azetidinyl, 2- and 3-oxetanyl, 2- and 3-thietanyl, 2- and 3-thietanyl-S-oxid (S-oxothietanyl), 2- and 3-thietanyl-S-dioxid (S-d ioxothietanyl), 2- and 3-pyrrolidinyl, 2- and 3-tetrahydrofuranyl, 1,3-dioxolan-2-yl, thiolan-2-yl, S-oxothiolan-2-yl, S-diox-othiolan-2-yl, 4- and 5-oxazolidinyl, 1,3-dioxan-2-yl, 1- and 3-thiopyran-2-yl, S-oxothiopyranyl, and S-dioxothiopyranyl.

The term “hetaryl” includes monocyclic 5- or 6-membered heteroaromatic radicals comprising as ring members 1, 2, or 3 heteroatoms selected from N, O and S. Examples of 5- or 6-membered heteroaromatic radicals include pyridyl, i.e. 2-, 3-, and 4-pyridyl, pyrimidinyl, i.e. 2-, 4- and 5-pyrimidinyl, pyrazinyl, pyridazinyl, i.e. 3- and 4-pyridazinyl, thienyl, i.e. 2- and 3-thienyl, furyl, i.e. 2- and 3-furyl, pyrrolyl, i.e. 1-, 2- and 3-pyrrolyl, oxazolyl, i.e. 2-, 4- and 5-oxazolyl, isoxazolyl, i.e. 3-, 4- and 5-isoxazolyl, thiazolyl, i.e. 2-, 3- and 5-thiazolyl, isothiazolyl, i.e. 3-, 4- and 5-isothiazolyl, pyrazolyl, i.e. 1-, 3-, 4- and 5-pyrazolyl, imidazolyl, i.e. 1-, 2-, 4- and 5-imidazolyl, oxadiazolyl, e.g. 2- and 5-[1,3,4]oxadiazolyl, thiadiazolyl, e.g. 1,3,4-thiadiazol-5-yl, 1,2,4-thiadia-zol-3-yl, triazolyl, e.g. 1,3,4-triazol-2-yl, and 1,2,4-triazol-3-yl.

The terms “heterocyclyolalkyl” and “hetarylalkyl” refer to heterocyclyl or hetaryl, resp., as defined above which are bound via a C₁-C₄-alkyl group, in particular a methyl group (=heterocy-clylmethyl or hetarylmethyl, resp.), to the remainder of the molecule.

With respect to the variables, the particularly preferred embodiments of the intermediates correspond to those of the compounds of the formula I.

In a particular embodiment, the variables of the compounds of the formula I have the following meanings, these meanings, both on their own and in combination with one another, being particular embodiments of the compounds of formula I.

In a preferred embodiment, the compounds I are present in form of a mixture of compounds I.A and I.B, wherein compound I.A with S-configuration in the W—Z-containing ring is present in an amount of more than 50% by weight, in particular of at least 70% by weight, more particularly of at least 85% by weight, specifically of at least 90% by weight, based on the total weight of compounds I.A and I.B.

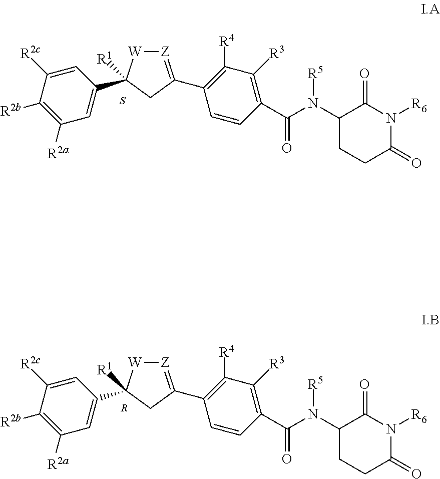

In one particularly preferred embodiment of the invention, the method comprises the step of contacting the plant, parts of it, its propagation material, the pests, their food supply, habitat or breeding grounds a pesticidally effective amount of a compound of formula I.A.

Compounds of formula I.A, and I.B, resp., can be obtained in enantiopure form by known separation methods, preferably by chiral chromatography. This is preferably applied to intermediate compounds of formula IIa.

In another preferred embodiment the glutarimide ring is present in form of a mixture of compounds I.a and I.b, wherein compound I.a (R-configuration in glutarimide) is present in an amount of more than 50% by weight, in particular of at least 70% by weight, more particularly of at least 85% by weight, specifically of at least 90% by weight, based on the total weight of compounds I.a and I.b.

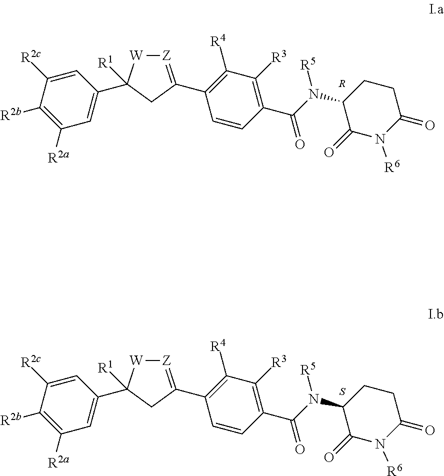

In another preferred embodiment the compounds of formula I are present in form of a mixture of stereoisomers as shown above, wherein compound I.Aa (S-configuration in W—Z-ring and R-configuration in glutarimide) is present in an amount of more than 50% by weight, in particular of at least 70% by weight, more particularly of at least 85% by weight, specifically of at least 90% by weight, based on the total weight of stereoisomers of formula I.

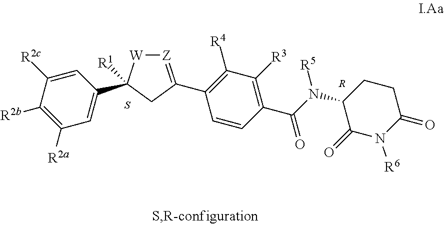

Accordingly, in a particularly preferred embodiment of the invention, the method comprises step of contacting the plant, parts of it, its propagation material, the pests, their food supply, habitat or breeding grounds a pesticidally effective amount of a compound of formula I.Aa.

Racemates of compounds of formula I consist of four stereoisomers I.Aa, I.Ab, I.Ba, and I.Bb. Accordingly isomer I.A consists of more than 50% by weight, in particular of at least 70% by weight, more particularly of at least 85% by weight, specifically of at least 90% by weight of two stereoisomers I.Aa and I.Ab.

Isomer I.a consists of more than 50% by weight, in particular of at least 70% by weight, more particularly of at least 85% by weight, specifically of at least 90% by weight of two stereoisomers I.Aa and I.Ba.

Preferably —W—Z═ in formula I is —O—N═; such compounds correspond to formula I.1.
In another embodiment W—Z in formula I is —CH₂—N═; such compounds correspond to formula I.2.
In another embodiment W—Z in formula I is —CH₂—CH═; such compounds correspond to formula I.3.

R^{2a} is preferably selected from F, Cl, Br, CF₃, and OCF₃.

R^{2b} and R^{2c} are independently preferably selected from H, F, Cl, Br, CF₃, and OCF₃.

Particularly preferred is each one of the following combinations of R^{2a}, R^{2b}, and R^{2c} wherein each line of Table A denotes a substitution pattern of the phenyl ring (“A”) bearing the R^{2a}, R^{2b}, and R^{2c} moieties.

TABLE A

| No. | R^{2a} | R^{2b} | R^{2c} |
|---|---|---|---|
| A-1 | F | F | H |
| A-2 | F | H | F |
| A-3 | F | F | F |
| A-4 | F | Cl | F |
| A-5 | F | Br | F |
| A-6 | F | H | Cl |
| A-7 | F | H | Br |
| A-8 | Cl | F | H |
| A-9 | Cl | H | Cl |
| A-10 | Cl | Cl | Cl |
| A-11 | Cl | F | Cl |
| A-12 | Cl | Br | Cl |
| A-13 | Cl | H | Br |
| A-14 | Br | F | H |
| A-15 | Br | H | Br |
| A-16 | Br | F | Br |
| A-17 | Br | Cl | Br |
| A-18 | CF₃ | H | H |
| A-19 | CF₃ | H | F |
| A-20 | CF₃ | H | Cl |
| A-21 | CF₃ | H | Br |
| A-22 | CF₃ | H | CF₃ |
| A-23 | CF₃ | F | F |
| A-24 | CF₃ | F | Cl |
| A-25 | CF₃ | Cl | Cl |
| A-26 | CF₃ | F | H |
| A-27 | OCF₃ | H | F |
| A-28 | OCF₃ | H | Cl |
| A-29 | OCF₃ | F | H |
| A-30 | OCF₃ | H | CF₃ |
| A-31 | OCF₃ | H | H |

Groups A-8, A-9, and A-11 are more preferred patterns in formula I compounds. A-11 is particularly preferred.

R³ and R⁴ are preferably halogen such as Cl and F, NO2, CN, CH₃, fluoromethyl such as CHF₂, CF₃, SCH₃, OCH₃. More preferably R⁴ is H, and R³ has one of the preferred meanings, particularly is Cl, or CH₃.

In another embodiment R³ and R⁴ together with the C-atoms they are bound to form a 5- or 6-membered saturated carbocyclic ring.

R⁵ is preferably H.

In one embodiment R⁶ is C₁-C₆-alkyl, C₁-C₄-haloalkyl, C₁-C₆-alkoxy, C₁-C₄-haloalkoxy, C₃-C₆-alkenyl, S(O)ₘN(R¹⁰)₂, or N(R¹⁰)₂, which groups are unsubstituted or substituted with OH, C₁-C₄-alkoxy, C(═O)OR^{a1}, C(═O)N(R^{a2})R^{a3}, CH═NOR^{a1}, or R⁶ is phenyl, benzyl, which rings are unsubstituted or substituted with halogen, C₁-C₄-alkyl, or C₁-C₄-haloalkyl, wherein R^{a1}, R^{a2}, R^{a3}, R¹⁰ are independently H, or C₁-C₄-alkyl.

Preferred embodiments relate to each of following compounds of formula I, wherein the variables are as defined in the outset and the preferred embodiments:

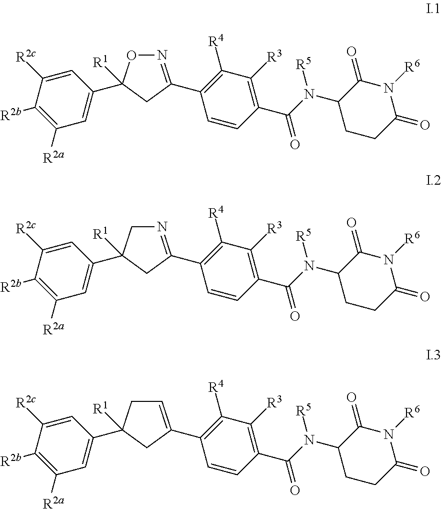

In particular with a view to their use, preference is given to the compounds of formula I com-piled in the tables below, which compounds correspond to formulae I.1, I.2, and I.3, resp., more preferably in configuration I.a, particularly in I.Aa. Each of the groups mentioned for a substituent in the tables is furthermore per se, independently of the combination in which it is mentioned, a particularly preferred aspect of the substituent in question.

Table 1: Compounds of formula I.1 in which R⁵ is H, R⁶ is CH₃, and the other variables for a compound correspond in each case to one row of Table B

Table 2: Compounds of formula I.2 in which R⁵ is H, R⁶ is CH₃, and the other variables for a compound correspond in each case to one row of Table B

Table 3: Compounds of formula I.3 in which R⁵ is H, R⁶ is CH₃, and the other variables for a compound correspond in each case to one row of Table B

Table 4: Compounds of formula I.1 in which R⁵ is H, R⁶ is C₂H₅, and the other variables for a compound correspond in each case to one row of Table B

Table 5: Compounds of formula I.2 in which R⁵ is H, R⁶ is C₂H₅, and the other variables for a compound correspond in each case to one row of Table B

Table 6: Compounds of formula I.3 in which R⁵ is H, R⁶ is C₂H₅, and the other variables for a compound correspond in each case to one row of Table B

Table 7: Compounds of formula I.1 in which R⁵ is H, R⁶ is CH₂CH₂CH₃, and the other variables for a compound correspond in each case to one row of Table B

Table 8: Compounds of formula I.2 in which R⁵ is H, R⁶ is CH₂CH₂CH₃, and the other variables for a compound correspond in each case to one row of Table B

Table 9: Compounds of formula I.3 in which R⁵ is H, R⁶ is CH₂CH₂CH₃, and the other variables for a compound correspond in each case to one row of Table B

Table 10: Compounds of formula I.1 in which R⁵ is H, R⁶ is CH₂(CH₃)₂, and the other variables for a compound correspond in each case to one row of Table B

Table 11: Compounds of formula I.2 in which R⁵ is H, R⁶ is CH₂(CH₃)₂, and the other variables for a compound correspond in each case to one row of Table B

Table 12: Compounds of formula I.3 in which R⁵ is H, R⁶ is CH₂(CH₃)₂, and the other variables for a compound correspond in each case to one row of Table B

Table 13: Compounds of formula I.1 in which R⁵ is H, R⁶ is CH₂CH₂(CH₃)₂, and the other variables for a compound correspond in each case to one row of Table B

Table 14: Compounds of formula I.2 in which R⁵ is H, R⁶ is CH₂CH₂(CH₃)₂, and the other variables for a compound correspond in each case to one row of Table B

Table 15: Compounds of formula I.3 in which R⁵ is H, R⁶ is CH₂CH₂(CH₃)₂, and the other variables for a compound correspond in each case to one row of Table B

Table 16: Compounds of formula I.1 in which R⁵ is H, R⁶ is CH₂CH═CH₂, and the other variables for a compound correspond in each case to one row of Table B

Table 17: Compounds of formula I.2 in which R⁵ is H, R⁶ is CH₂CH═CH₂, and the other variables for a compound correspond in each case to one row of Table B

Table 18: Compounds of formula I.3 in which R⁵ is H, R⁶ is CH₂CH═CH₂, and the other variables for a compound correspond in each case to one row of Table B

Table 19: Compounds of formula I.1 in which R⁵ is H, R⁶ is CH₂CH₂F, and the other variables for a compound correspond in each case to one row of Table B

Table 20: Compounds of formula I.2 in which R⁵ is H, R⁶ is CH₂CH₂F, and the other variables for a compound correspond in each case to one row of Table B

Table 21: Compounds of formula I.3 in which R⁵ is H, R⁶ is CH₂CH₂F, and the other variables for a compound correspond in each case to one row of Table B

Table 22: Compounds of formula I.1 in which R⁵ is H, R⁶ is CH₂CHF₂, and the other variables for a compound correspond in each case to one row of Table B

Table 23: Compounds of formula I.2 in which R⁵ is H, R⁶ is CH₂CHF₂, and the other variables for a compound correspond in each case to one row of Table B

Table 24: Compounds of formula I.3 in which R⁵ is H, R⁶ is CH₂CHF₂, and the other variables for a compound correspond in each case to one row of Table B

Table 25: Compounds of formula I.1 in which R⁵ is H, R⁶ is CH₂CF₃, and the other variables for a compound correspond in each case to one row of Table B

Table 26: Compounds of formula I.2 in which R⁵ is H, R⁶ is CH₂CF₃, and the other variables for a compound correspond in each case to one row of Table B

Table 27: Compounds of formula I.3 in which R⁵ is H, R⁶ is CH₂CF₃, and the other variables for a compound correspond in each case to one row of Table B

Table 28: Compounds of formula I.1 in which R⁵ is H, R⁶ is CH₂CH₂CF₃, and the other variables for a compound correspond in each case to one row of Table B

Table 29: Compounds of formula I.2 in which R⁵ is H, R⁶ is CH₂CH₂CF₃, and the other variables for a compound correspond in each case to one row of Table B

Table 30: Compounds of formula I.3 in which R⁵ is H, R⁶ is CH₂CH₂CF₃, and the other variables for a compound correspond in each case to one row of Table B

Table 31: Compounds of formula I.1 in which R⁵ is H, R⁶ is CH₂CH₂OH, and the other variables for a compound correspond in each case to one row of Table B

Table 32: Compounds of formula I.2 in which R⁵ is H, R⁶ is CH₂CH₂OH, and the other variables for a compound correspond in each case to one row of Table B

Table 33: Compounds of formula I.3 in which R⁵ is H, R⁶ is CH₂CH₂OH, and the other variables for a compound correspond in each case to one row of Table B

Table 34: Compounds of formula I.1 in which R⁵ is H, R⁶ is CH₂CH₂OCH₃, and the other variables for a compound correspond in each case to one row of Table B

Table 35: Compounds of formula I.2 in which R⁵ is H, R⁶ is CH₂CH₂OCH₃, and the other variables for a compound correspond in each case to one row of Table B

Table 36: Compounds of formula I.3 in which R⁵ is H, R⁶ is CH₂CH₂OCH₃, and the other variables for a compound correspond in each case to one row of Table B

Table 37: Compounds of formula I.1 in which R⁵ is H, R⁶ is c-C₃H₅, and the other variables for a compound correspond in each case to one row of Table B

Table 38: Compounds of formula I.2 in which R⁵ is H, R⁶ is c-C₃H₅, and the other variables for a compound correspond in each case to one row of Table B

Table 39: Compounds of formula I.3 in which R⁵ is H, R⁶ is c-C₃H₅, and the other variables for a compound correspond in each case to one row of Table B

Table 40: Compounds of formula I.1 in which R⁵ is H, R⁶ is CH₂C₆H₅, and the other variables for a compound correspond in each case to one row of Table B

Table 41: Compounds of formula I.2 in which R⁵ is H, R⁶ is CH₂C₆H₅, and the other variables for a compound correspond in each case to one row of Table B

Table 42: Compounds of formula I.3 in which R⁵ is H, R⁶ is CH₂C₆H₅, and the other variables for a compound correspond in each case to one row of Table B

Table 43: Compounds of formula I.1 in which R⁵ is H, R⁶ is OCH₃, and the other variables for a compound correspond in each case to one row of Table B

Table 44: Compounds of formula I.2 in which R⁵ is H, R⁶ is OCH₃, and the other variables for a compound correspond in each case to one row of Table B

Table 45: Compounds of formula I.3 in which R⁵ is H, R⁶ is OCH₃, and the other variables for a compound correspond in each case to one row of Table B

Table 46: Compounds of formula I.1 in which R⁵ is H, R⁶ is OCH₂CH₃, and the other variables for a compound correspond in each case to one row of Table B

Table 47: Compounds of formula I.2 in which R⁵ is H, R⁶ is OCH₂CH₃, and the other variables for a compound correspond in each case to one row of Table B

Table 48: Compounds of formula I.3 in which R⁵ is H, R⁶ is OCH₂CH₃, and the other variables for a compound correspond in each case to one row of Table B

Table 49: Compounds of formula I.1 in which R⁵ is H, R⁶ is OCH₂CF₃, and the other variables for a compound correspond in each case to one row of Table B

Table 50: Compounds of formula I.2 in which R⁵ is H, R⁶ is OCH₂CF₃, and the other variables for a compound correspond in each case to one row of Table B

Table 51: Compounds of formula I.3 in which R⁵ is H, R⁶ is OCH₂CF₃, and the other variables for a compound correspond in each case to one row of Table B

Table 52: Compounds of formula I.1 in which R⁵ is H, R⁶ is SO₂N(CH₃)₂, and the other variables for a compound correspond in each case to one row of Table B

Table 53: Compounds of formula I.2 in which R⁵ is H, R⁶ is SO₂N(CH₃)₂, and the other variables for a compound correspond in each case to one row of Table B

Table 54: Compounds of formula I.3 in which R⁵ is H, R⁶ is SO₂N(CH₃)₂, and the other variables for a compound correspond in each case to one row of Table B

Table 55: Compounds of formula I.1 in which R⁵ is H, R⁶ is N(CH₃)₂, and the other variables for a compound correspond in each case to one row of Table B

Table 56: Compounds of formula I.2 in which R⁵ is H, R⁶ is N(CH₃)₂, and the other variables for a compound correspond in each case to one row of Table B

Table 57: Compounds of formula I.3 in which R⁵ is H, R⁶ is N(CH₃)₂, and the other variables for a compound correspond in each case to one row of Table B

Table 58: Compounds of formula I.1 in which R⁵ is H, R⁶ is CH₂C(═O)OCH₃, and the other variables for a compound correspond in each case to one row of Table B

Table 59: Compounds of formula I.2 in which R⁵ is H, R⁶ is CH₂C(═O)OCH₃, and the other variables for a compound correspond in each case to one row of Table B

Table 60: Compounds of formula I.3 in which R⁵ is H, R⁶ is CH₂C(═O)OCH₃, and the other variables for a compound correspond in each case to one row of Table B

Table 61: Compounds of formula I.1 in which R⁵ is H, R⁶ is CH₂C(═O)OC₂H₅, and the other variables for a compound correspond in each case to one row of Table B

Table 62: Compounds of formula I.2 in which R⁵ is H, R⁶ is CH₂C(═O)OC₂H₅, and the other variables for a compound correspond in each case to one row of Table B

Table 63: Compounds of formula I.3 in which R⁵ is H, R⁶ is CH₂C(═O)OC₂H₅, and the other variables for a compound correspond in each case to one row of Table B

TABLE B

| No. | R^{2a}, R^{2b}, R^{2c} | R¹ | R³ | R⁴ |
|---|---|---|---|---|
| I-1 | A-8 | CF₃ | CH₃ | H |
| I-2 | A-9 | CF₃ | CH₃ | H |
| I-3 | A-11 | CF₃ | CH₃ | H |
| I-4 | A-8 | CF₃ | CH₃ | H |
| I-5 | A-9 | CF₃ | CH₃ | H |
| I-6 | A-11 | CF₃ | CH₃ | H |
| I-7 | A-8 | CF₃ | CH₃ | H |
| I-8 | A-9 | CF₃ | CH₃ | H |
| I-9 | A-11 | CF₃ | CH₃ | H |
| I-10 | A-8 | CF₂Cl | CH₃ | H |
| I-11 | A-9 | CF₂Cl | CH₃ | H |
| I-12 | A-11 | CF₂Cl | CH₃ | H |
| I-13 | A-8 | CF₂Cl | CH₃ | H |
| I-14 | A-9 | CF₂Cl | CH₃ | H |
| I-15 | A-11 | CF₂Cl | CH₃ | H |
| I-16 | A-8 | CF₂Cl | CH₃ | H |
| I-17 | A-9 | CF₂Cl | CH₃ | H |
| I-18 | A-11 | CF₂Cl | CH₃ | H |
| I-19 | A-8 | CF₃ | Cl | H |
| I-20 | A-9 | CF₃ | Cl | H |
| I-21 | A-11 | CF₃ | Cl | H |
| I-22 | A-8 | CF₃ | Cl | H |
| I-23 | A-9 | CF₃ | Cl | H |
| I-24 | A-11 | CF₃ | Cl | H |
| I-25 | A-8 | CF₃ | Cl | H |
| I-26 | A-9 | CF₃ | Cl | H |
| I-27 | A-11 | CF₃ | Cl | H |
| I-28 | A-8 | CF₂Cl | Cl | H |
| I-29 | A-9 | CF₂Cl | Cl | H |
| I-30 | A-11 | CF₂Cl | Cl | H |
| I-31 | A-8 | CF₂Cl | Cl | H |
| I-32 | A-9 | CF₂Cl | Cl | H |
| I-33 | A-11 | CF₂Cl | Cl | H |
| I-34 | A-8 | CF₂Cl | Cl | H |
| I-35 | A-9 | CF₂Cl | Cl | H |
| I-36 | A-11 | CF₂Cl | Cl | H |
| I-37 | A-8 | CF₃ | F | H |
| I-38 | A-9 | CF₃ | F | H |
| I-39 | A-11 | CF₃ | F | H |
| I-40 | A-8 | CF₃ | F | H |
| I-41 | A-9 | CF₃ | F | H |
| I-42 | A-11 | CF₃ | F | H |
| I-43 | A-8 | CF₃ | F | H |
| I-44 | A-9 | CF₃ | F | H |
| I-45 | A-11 | CF₃ | F | H |
| I-46 | A-8 | CF₂Cl | F | H |
| I-47 | A-9 | CF₂Cl | F | H |
| I-48 | A-11 | CF₂Cl | F | H |
| I-49 | A-8 | CF₂Cl | F | H |
| I-50 | A-9 | CF₂Cl | F | H |
| I-51 | A-11 | CF₂Cl | F | H |
| I-52 | A-8 | CF₂Cl | F | H |
| I-53 | A-9 | CF₂Cl | F | H |
| I-54 | A-11 | CF₂Cl | F | H |
| I-55 | A-8 | CF₃ | CF₃ | H |
| I-56 | A-9 | CF₃ | CF₃ | H |
| I-57 | A-11 | CF₃ | CF₃ | H |
| I-58 | A-8 | CF₃ | CF₃ | H |
| I-59 | A-9 | CF₃ | CF₃ | H |
| I-60 | A-11 | CF₃ | CF₃ | H |
| I-61 | A-8 | CF₃ | CF₃ | H |
| I-62 | A-9 | CF₃ | CF₃ | H |
| I-63 | A-11 | CF₃ | CF₃ | H |
| I-64 | A-8 | CF₂Cl | CF₃ | H |
| I-65 | A-9 | CF₂Cl | CF₃ | H |
| I-66 | A-11 | CF₂Cl | CF₃ | H |
| I-67 | A-8 | CF₂Cl | CF₃ | H |
| I-68 | A-9 | CF₂Cl | CF₃ | H |
| I-69 | A-11 | CF₂Cl | CF₃ | H |
| I-70 | A-8 | CF₂Cl | CF₃ | H |
| I-71 | A-9 | CF₂Cl | CF₃ | H |
| I-72 | A-11 | CF₂Cl | CF₃ | H |

A preferred embodiment relates to compounds in configuration I.a, particularly I.Aa, which correspond to formulae I.1, I.2, or I.3, particularly to formula I.1. In such compounds ring “A” is substituted by patterns A-8, A-9, or A-11, R¹ is CF₂Cl or CF₃, R³ is Cl or CH₃, R⁴ and R⁵ are H, and R⁶ is CH₃, OC₂H₅, CH₂CH₂CH₃, CH(CH₃)₂, CH₂CH(CH₃)₂, CH₂CH═CH₂, CH₂CH₂F, CH₂CHF₂, CH₂CF₃, CH₂CH₂CF₃, c-C₃H₅, CH₂CH₂OH, CH₂CH₂OCH₃, CH₂C₆H₅, CH₂C(═O)OCH₃, CH₂C(═O)OC₂H₅, OCH₃, OC₂H₅, OCH₂CF₃, SO₂N(CH₃)₂, or N(CH₃)₂.

A preferred embodiment relates to compounds in configuration I.a, particularly I.Aa, which correspond to formulae I.1, I.2, or I.3, particularly to formula I.1. In such compounds ring “A” is substituted by patterns A-8, A-9, or A-11, R¹ is CF₃, R³ is Cl or CH₃, R⁴ is H, or R³ and R⁴ together form a C₃-carbon chain, R⁵ is H, and R⁶ is H, CH₃, C₂H₅, CH₂CH(CH₃)₂, CH₂CH₂F, CH₂CHF₂, c-C₃H₅, OCH₃, or N(CH₃)₂.

More preferred embodiments relate to compounds in configuration I.a, particularly I.Aa, which correspond to formulae I.1, I.2, or I.3, particularly to formula I.1. In such compounds ring “A” is substituted by patterns A-8, A-9, or A-11, R¹ is CF₃, R³ is Cl or CH₃, R⁴ and R⁵ are H, and R⁶ is C₁-C₄-alkyl, C₁-C₃-haloalkyl, C₃-C₄-cycloalkyl, C₁-C₃-alkoxy, C₁-C₃-haloalkoxy, C₁-C₄-alkylamino, and di-C₁-C₄-alkylamino. Particularly preferred compounds are selected from following compounds of formula I.1, preferably in at least 85% by weight of the given isomer:

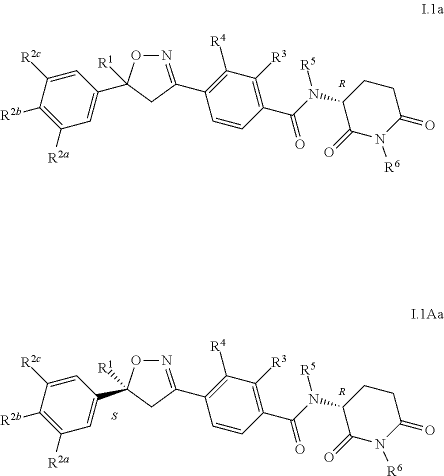

| No. | R¹ | R^{2a} | R^{2b} | R^{2c} | R³ | R⁴ | R⁵ | R⁶ | Isomer |
|---|---|---|---|---|---|---|---|---|---|
| I-1-13 | CF₃ | Cl | F | Cl | Cl | H | H | CH₃ | I.1Aa |
| I-1-19 | CF₃ | Cl | H | Cl | Cl | H | H | CH₃ | I.1a |
| I-1-20 | CF₃ | Cl | F | Cl | Cl | H | H | N(CH₃)₂ | I.1Aa |
| I-1-21 | CF₃ | Cl | F | Cl | Cl | H | H | CH₂CH₃ | I.1Aa |
| I-1-24 | CF₃ | Cl | F | Cl | Cl | H | H | CH₂CHF₂ | I.1Aa |
| I-1-25 | CF₃ | Cl | F | Cl | Cl | H | H | c-C₃H₅ | I.1Aa |
| I-1-28 | CF₃ | Cl | H | Cl | CH₃ | H | H | CH₃ | I.1a |
| I-1-32 | CF₃ | Cl | F | Cl | Cl | H | H | CH₃ | I.1A |
| I-1-40 | CF₃ | Cl | F | Cl | Cl | H | H | OCH₃ | I.1Aa |
| I-1-47 | CF₃ | Cl | H | Cl | CH₃ | H | H | CH(CH₃)₂ | I.1a |
| I-1-48 | CF₃ | Cl | H | Cl | Cl | H | H | CH(CH₃)₂ | I.1a |
| I-1-51 | CF₃ | Cl | H | Cl | CH₃ | H | H | CH₂CH₃ | I.1a |
| I-1-52 | CF₃ | Cl | H | Cl | Cl | H | H | CH₂CH₃ | I.1a |
| I-1-76 | CF₃ | Cl | F | Cl | Cl | H | H | OCH₂CF₃ | I.1Aa |
| I-1-80 | CF₃ | Cl | F | Cl | Cl | H | H | CH₂CH(CH₃)₂ | I.1Aa |
| I-1-82 | CF₃ | Cl | F | Cl | Cl | H | H | OCH₂CH₃ | I.1Aa |

The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protec-tive colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emusifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-substituted fatty acid amides, amine oxides, esters, sugar-based surfactants, polymeric surfactants. Suitable cationic surfactants are quaternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions of the invention in a weight ratio of 1:100 to 100:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grapefruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds of the invention are especially suitable for efficiently combating animal pests e.g. arthropods, and nematodes including:

insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;

Aphids, e.g. Acyrthosnohon pisum, Aphis spp., Myzus persicae, Rhopalosnohum spp., Schi-zaphIS graminum, Megoura viciae;

Coccoidea, e.g. Aonidiella aurantia, Ferrisia virgate;

Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epllachna spp.;

Flies, e.g. Delia spp., Ceratitis capitate, Bactrocera spp., Liriomyza spp.;

Lepidoptera, e.g. Helicoverpa spp., Heliothis virescens, Lobesia botrana, Ostrinia nubllalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressaks, Anticarsia gemmatalis, Agrotis ipsilon, Chrysodeixis includens;

Thrips, e.g. Frankliniella spp., Thrips spp., Dichromothrips corbetti;

True bugs, e.g. Lygus spp., Stink bugs such as Euschistus spp., Halyomorpha halys, Nezara viridula, Piezodorus guildinii, Dichelops furcatus;

Whiteflies, e.g. Trialeurodes vaporariorum, Bemisia spp.;

Anthropods of class Arachnida (Mites), e.g. Penthaleus major, Tetranychus spp.;

Nematodes, e.g. Heterodera glycines, Meloidogyne sp., Pratylenchus spp., Caenorhabditis elegans.

### EXAMPLES

### A. Preparation Examples

With appropriate modification of the starting materials, the procedures given in the synthesis description were used to obtain further compounds I. The compounds obtained in this manner are listed in the table that follows, together with physical data.

The products shown below were characterized by melting point determination, by NMR spectroscopy or by the masses ([m/z]) or retention time (RT; [min.]) determined by HPLC-MS or HPLC spectrometry.

HPLC-MS=high performance liquid chromatography-coupled mass spectrometry;

HPLC method A: HPLC Phenomenex Kinetex 1.7 μm XB—C18 100A, 50×2.1 mm″, Mobile Phase: A: water+0.1% TFA; B:CAN; Temperature: 60° C.; Gradient: 5% B to 100% B in 1.50 min; 100% B 0.25 min; Flow: 0.8 ml/min to 1.0 ml/min in 1.51 min; MS method: ESI positive; Mass range (m/z): 100-700″.

HPLC method B: HPLC method: Phenomenex Kinetex 1.7 μm XB—C18 100A; 50×2.1 mm; mobile phase: A: water+0.1% trifluoroacetic acid (TFA); B: acetonitrile; gradient: 5-100% B in 1.50 minutes; 100% B 0.25 min; flow: 0.8-1.0 ml/min in 1.51 minutes at 60° C. MS: ESI positive, m/z 100-1400.

The synthesis of 2-chloro-4-[5-(3,5-dichloro-4-fluoro-phenyl)-5-(trifluoromethyl)-4H-isoxazol-3-yl]benzoic acid and related carboxylic acids was performed in analogy to WO 2017/050922, WO 2013/026695, and WO 2016/102482.

### Example 1: 2-chloro-4[5-(3,5-dichloro-4-fluoro-phenyl)-5-(trifluoromethyl)-4H-isoxazol-3-yl]-N-[(3R)-1-methyl-2,6-dioxo-3-piperidyl]benzamide [Compound 1-1 of table C]

Step 1: 2-chloro-4[5-(3,5-dichloro-4-fluoro-phenyl)-5-(trifluoromethyl)-4H-isoxazol-3-yl]-N-[(3R)-1-methyl-2,6-dioxo-3-piperidyl]benzamide: To a solution of 479 mg 2-chloro-4[5-(3,5-di-chloro-4-fluoro-phenyl)-5-(trifluoromethyl)-4H-isoxazol-3-yl]benzoic acid, 281 mg (3R)-3-amino-1-methyl-piperidine-2,6-dionehydrobromide (commercial), 587 mg PyBroP in 5 mL dichloromethane was added 0.75 mL diisopropyl ehtylamine at 20-25° C. and stirred for about 14 h. The mixture was concentrated at reduced pressure and purified via flash chromatography on silica gel to obtain the title compound (413 mg, 68%). HPLC-MS: 1.342 min; m/z=581.8

### Example 2: 4-[5-(3,5-dichlorophenyl)-5-(trifluoromethyl)-4H-isoxazol-3-yl]-N-[(3R)-1-(2,2-difluoroethyl)-2,6-dioxo-3-piperidyl]-2-methyl-benzamide [Compound 1-18 of table C]

Step 1: Benzyl N-[(3R)-1-(2,2-difluoroethyl)-2,6-dioxo-3-piperidyl]carbamate: To a solution of N-carbobenzoxy-D-glutamic acid (25.2 g, 89.4 mmol; commercial) and iPr₂NEt (19.0 mL, 112 mmol, 1.25 equiv) dissolved in CH₂Cl₂ (250 mL) at ambient temperature was added 1,1′-carbonyldiimidazole (18.0 g, 112 mmol, 1.25 equiv) portionwise and the resulting mixture was stirred at that temperature for 4 h. To this mixture, 2,2-difluoroethylamine hydrochloride (11.6 g, 1.1 equiv) was added and stirring was continued for another 18 h. After that time, another portion of 1,1′-carbonyldiimidazole (18.0 g, 112 mmol, 1.25 equiv) was added before heating the mixture at reflux for 3 h. The resulting reaction mixture was allowed to cool to ambient temperature, and the organic phase washed with HCl solution (10% in H₂O, 1×200 mL) and NaCl solution (sat. aqueous, 1×100 mL), dried over Na₂SO₄, filtered and concentrated under reduced pressure. Purification by column chromatography (EtOAc/cyclohexane 0:100 to 100:0, gradient) afforded the title compound (25.0 g, 86%) in an enantiomeric ratio of 77:23. If desired, the enantiomeric ratio could be further enhanced via recrystallization from MTBE. HPLC-MS: 0.963 min; m/z=326.8.

Step 2: (3R)-3-amino-1-(2,2-difluoroethyl)piperidine-2,6-dione hydrobromide: Benzyl N-[(3R)-1-(2,2-difluoroethyl)-2,6-dioxo-3-piperidyl]carbamate (10.7 g, 32.7 mmol) was added to HBr, 33% in AcOH (100 mL) at ambient temperature and the resulting reaction mixture was stirred at that temperature for 3 h. After that time, the mixture was poured on cold H₂O (300 mL), the aqueous phase was washed with CH₂Cl₂ (1×300 mL) and concentrated under reduced pressure. Residual water was removed by co-distillation with EtOAc to afford the title compound (8.96 g, quantitative). HPLC-MS: 0.232 min; m/z=192.8.

Step 3: 4-[5-(3,5-dichlorophenyl)-5-(trifluoromethyl)-4H-isoxazol-3-yl]-N-[(3R)-1-(2,2-difluoro-ethyl)-2,6-dioxo-3-piperidyl]-2-methyl-benzamide [Compound 1-18 of table C]: From the above (3R)-3-amino-1-(2,2-difluoroethyl)piperidine-2,6-dione hydrobromide the title compound was synthesized in analogy as described for example 1 (step 1).

HPLC-MS: 1.353 min; m/z=592.1.

### Example 3: 4-[5-(3,5-dichlorophenyl)-5-(trifluoromethyl)-4H-isoxazol-3-yl]-N-[(3R)-1-(2,2-di-fluoroethyl)-2,6-dioxo-3-piperidyl]-2-methyl-benzamide [Compound 3-1 of table C]

Step 1: (3R)-3-amino-1-(methylamino)piperidine-2,6-dione hydrochloride: To a solution of benzyl N-[(3R)-3-(benzyloxycarbonylamino)-2,6-dioxo-1-piperidyl]-N-methyl-carbamate (1.40 g, 3.29 mmol; obtained analogously as described in example 2, step 1) dissolved in THF (10 mL) at ambient temperature under an atmosphere of N₂ was added HCl solution, 1.0 M in H₂O (4.6 mL, 4.6 mmol), followed by Pd(OH)₂, 20 wt % on carbon (139 mg, 0.197 mmol). The flask was purged with H₂ using a gas burette and the resulting suspension was vigorously stirred under an atmosphere of H₂ for 3 h. After that time, the flask was purged with N₂ and the resulting reaction mixture filtered through a short plug of Celite, eluting with MeOH. The filtrate was concentrated under reduced pressure, the residue dried via azeotropical distillation with EtOAc, to afford the title compound (0.492 g, 77%) in crude form which was used in the next step without further purification. HPLC-MS: 0.160 min; m/z=158.1.

Step 3: 4-[5-(3,5-dichlorophenyl)-5-(trifluoromethyl)-4H-isoxazol-3-yl]-N-[(3R)-1-(2,2-difluoro-ethyl)-2,6-dioxo-3-piperidyl]-2-methyl-benzamide [Compound 3-1 of table C]: From the above (3R)-3-amino-1-(methylamino)piperidine-2,6-dione hydrochloride the title compound was synthesized in analogy as described for Example 1 (step 1). HPLC-MS: 1.244 min; m/z=557.8.

TABLE C
Compounds of formula I.1 (R¹ = CF₃, R⁵ = H)
SR-I

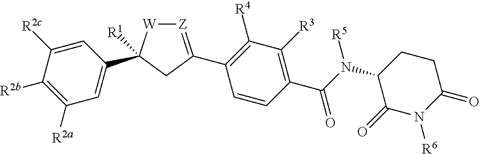

racR-I

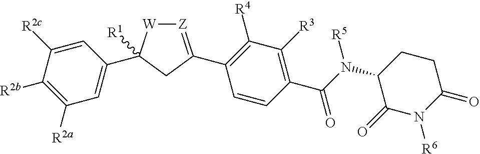

RR-I

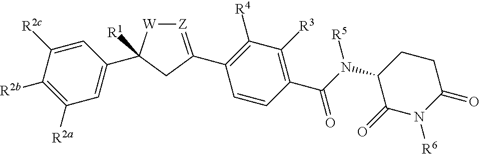

| No. | R^{2a} | R^{2b} | R^{2c} | R³ | R⁴ | W—Z | Formula | R⁶ | HPLC Method | HPLC Rt [min] | M + H [m/z] |
|---|---|---|---|---|---|---|---|---|---|---|---|
| 1-1 | Cl | F | Cl | Cl | H | O—N | racR-I | CH₃ | B | 1.337 | 581.8 |
| 1-2 | Cl | F | Cl | Cl | H | O—N | racR-I | C₂H₅ | B | 1.381 | 595.8 |
| 1-3 | Cl | F | Cl | Cl | H | O—N | racR-I | c-C₃H₅ | B | 1.355 | 607.7 |
| 1-4 | Cl | F | Cl | Cl | H | O—N | racR-I | H | B | 1.277 | 567.7 |
| 1-5 | Cl | F | Cl | Cl | H | O—N | racR-I | N(CH₃)₂ | B | 1.313 | 609.9 |
| 1-6 | Cl | F | Cl | Cl | H | O—N | racR-I | CH₂CHF₂ | B | 1.381 | 631.9 |
| 1-7 | Cl | F | Cl | Cl | H | O—N | racR-I | OCH₃ | A | 1.319 | 597.8 |
| 1-8 | Cl | H | Cl | Cl | H | O—N | racR-I | CH₃ | A | 1.325 | 563.8 |
| 1-9 | Cl | H | Cl | CH₃ | H | O—N | racR-I | CH₃ | A | 1.312 | 542.0 |
| 1-10 | Cl | F | Cl | CH₃ | H | O—N | racR-I | CH₃ | A | 1.323 | 559.9 |
| 1-11 | Cl | H | Cl | Cl | H | O—N | racR-I | C₂H₅ | A | 1.355 | 577.8 |
| 1-12 | Cl | H | Cl | CH₃ | H | O—N | racR-I | C₂H₅ | A | 1.344 | 556.1 |
| 1-13 | Cl | F | Cl | CH₃ | H | O—N | racR-I | C₂H₅ | A | 1.352 | 574.1 |
| 1-14 | Cl | H | Cl | CH₃ | H | O—N | SR-I | CH₃ | A | 1.344 | 541.9 |
| 1-15 | Cl | H | Cl | CH₃ | H | O—N | SR-I | C₂H₅ | A | 1.356 | 555.9 |
| 1-16 | Cl | F | Cl | CH₃ | H | O—N | racR-I | CH₂CHF₂ | A | 1.361 | 610.0 |
| 1-17 | Cl | H | Cl | Cl | H | O—N | racR-I | CH₂CHF₂ | A | 1.363 | 612.0 |
| 1-18 | Cl | H | Cl | CH₃ | H | O—N | racR-I | CH₂CHF₂ | A | 1.353 | 592.1 |
| 1-19 | Cl | F | Cl | Cl | H | O—N | racR-I | CH₂CH₂F | B | 1.349 | 612.0 |
| 1-20 | Cl | F | Cl | CH₃ | H | O—N | racR-I | CH₂CH₂F | A | 1.337 | 592.1 |
| 1-21 | Cl | H | Cl | Cl | H | O—N | racR-I | CH₂CH₂F | A | 1.339 | 596.0 |
| 1-22 | Cl | H | Cl | CH₃ | H | O—N | racR-I | CH₂CH₂F | A | 1.330 | 574.0 |
| 1-23 | Cl | F | Cl | CH₃ | H | O—N | SR-I | C₂H₅ | A | 1.350 | 573.9 |
| 1-24 | Cl | H | Cl | CH₃ | H | O—N | racR-I | CH₂-c-C₃H₅ | A | 1.384 | 582.1 |
| 1-25 | Cl | H | Cl | Cl | H | O—N | racR-I | CH₂-c-C₃H₅ | A | 1.394 | 602.0 |
| 1-26 | Cl | F | Cl | CH₃ | H | O—N | racR-I | CH₂-c-C₃H₅ | A | 1.390 | 600.1 |
| 1-27 | Cl | F | Cl | Cl | H | O—N | racR-I | CH₂-c-C₃H₅ | A | 1.400 | 620.0 |
| 1-28 | Cl | F | Cl | Cl | H | O—N | racR-I | CH₂CH(CH₃)₂ | A | 1.425 | 623.7 |
| 1-29 | Cl | F | Cl | CH₃ | H | O—N | racR-I | CH₂CH(CH₃)₂ | A | 1.415 | 601.9 |
| 1-30 | Cl | H | Cl | Cl | H | O—N | racR-I | CH₂CH(CH₃)₂ | A | 1.418 | 605.8 |
| 1-31 | Cl | H | Cl | CH₃ | H | O—N | racR-I | CH₂CH(CH₃)₂ | A | 1.408 | 584.4 |
| 1-32 | Cl | F | Cl | Cl | H | O—N | racR-I | NHCH₃ | A | 1.279 | 596.6 |
| 1-33 | Cl | F | Cl | CH₃ | H | O—N | racR-I | NHCH₃ | A | 1.284 | 575.0 |
| 1-34 | Cl | H | Cl | Cl | H | O—N | racR-I | NHCH₃ | A | 1.283 | 579.0 |
| 1-35 | Cl | H | Cl | CH₃ | H | O—N | racR-I | NHCH₃ | A | 1.270 | 557.1 |
| 2-1 | Cl | H | Cl | —CH₂—CH₂—CH₂— | | CH₂—N | racR-I | NHCH₃ | A | 1.245 | 580.9 |
| 3-1 | Cl | F | H | Cl | H | CH₂—CH | racR-I | NHCH₃ | A | 1.244 | 557.8 |

II. Evaluation of Pesticidal Activity:

The activity of the compounds of formula I of the present invention can be demonstrated and evaluated by the following biological test.

B.1 Diamond Back Moth (Plutella xylostella)

The active compound was dissolved at the desired concentration in a mixture of 1:1 (vol:vol) distilled water:aceteone. Surfactant (Kinetic HV) was added at a rate of 0.01% (vol/vol). The test solution was prepared at the day of use.

Leaves of cabbage were dipped in test solution and air-dried. Treated leaves were placed in petri dishes lined with moist filter paper and inoculated with ten 3^{rd} instar larvae. Mortality was recorded 72 hours after treatment. Feeding damages were also recorded using a scale of 0-100%.

In this test, compounds 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 2-1, and 3-1, resp., at 300 ppm showed over 75% mortality in comparison with untreated controls.

B.2 Green Peach Aphid (Myzus persicae)

For evaluating control of green peach aphid (Myzus persicae) through systemic means the test unit consisted of 96-well-microtiter plates containing liquid artificial diet under an artificial mem brane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, compounds 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-33, 1-34, 1-35, 2-1, and 3-1, resp., at 2500 ppm showed over 75% mortality in comparison with untreated controls.

B.3 Vetch aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5-8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed.

In this test, compounds 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-33, 1-34, 1-35, 2-1, and 3-1, resp., at 2500 ppm showed over 75% mortality in comparison with untreated controls.

B.4 Tobacco Budworm (Heliothis Virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications.

After application, microtiter plates were incubated at about 28±1° C. and about 80±5% relative humidity for 5 days. Egg and larval mortality was then visually assessed.

In this test, compounds 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-33, 1-34, 1-35, 2-1, and 3-1, resp., at 2500 ppm showed over 75% mortality in comparison with untreated controls.

B.5 Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 25±1° C. and about 75±5% relative humidity for 5 days. Egg and larval mortality was then visually assessed.

In this test, compounds 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-33, 1-34, 1-35, 2-1, and 3-1, resp., at 2500 ppm showed over 75% mortality in comparison with untreated controls.

B.7 Orchid thrips (Dichromothrips Corbetti)

Dichromothrips corbetti adults used for bioassay were obtained from a colony maintained continuously under laboratory conditions. For testing purposes, the test compound is diluted in a 1:1 mixture of acetone:water (vol:vol), plus Kinetic HV at a rate of 0.01% v/v.

Thrips potency of each compound was evaluated by using a floral-immersion technique. All petals of individual, intact orchid flowers were dipped into treatment solution and allowed to dry in Petri dishes. Treated petals were placed into individual re-sealable plastic along with about 20 adult thrips. All test arenas were held under continuous light and a temperature of about 28° C. for duration of the assay. After 3 days, the numbers of live thrips were counted on each petal. The percent mortality was recorded 72 hours after treatment.

In this test, compounds 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 2-1, and 3-1, resp., at 300 ppm showed over 75% mortality in comparison with untreated controls.

B.8 Rice Green Leafhopper (Nephotettix Virescens)

Rice seedlings were cleaned and washed 24 hours before spraying. The active compounds were formulated in 1:1 acetone:water (vol:vol), and 0.01% vol/vol surfactant (Kinetic HV) was added. Potted rice seedlings were sprayed with 5-6 ml test solution, air dried, covered with Mylar cages and inoculated with 10 adults. Treated rice plants were kept at about 28-29° C. and relative humidity of about 50-60%. Percent mortality was recorded after 72 hours.

In this test, compounds 1-1, 1-2, 1-3, 1-4, 1-5, 1-6, 1-8, 1-9, 1-10, 1-11, 1-12, 1-13, 1-14, 1-15, I-1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, and 2-1, resp., at 300 ppm showed over 75% mortality in comparison with untreated controls.

B.9 Red Spider Mite (Tetranychus kanzawai)

The active compound was dissolved at the desired concentration in a mixture of 1:1 (vol:vol) distilled water:acetone. Add surfactant (Kinetic HV) was added at a rate of 0.01% (vol/vol). The test solution was prepared at the day of use.

Potted cowpea beans of 4-5 days of age were cleaned with tap water and sprayed with 1-2 ml of the test solution using air driven hand atomizer. The treated plants were allowed to air dry and afterwards inoculated with 30 or more mites by clipping a cassava leaf section from rearing population. Treated plants were placed inside a holding room at about 25-27° C. and about 50-60% relative humidity. Percent mortality was assessed 72 hours after treatment.

In this test, compounds 1-1, 1-2, 1-3, 1-4, 1-5, 1-6, 1-8, 1-9, 1-10, 1-11, 1-12, 1-13, 1-14, 1-15, 1-16, 1-18, 1-19, 1-20, 1-21, 1-22, 1-23, 1-24, 1-25, 1-26, 1-27, 1-28, 1-29, 1-31, 1-32, 1-33, 1-34, 1-35, 2-1, and 3-1, resp., at 300 ppm showed over 75% mortality in comparison with untreated controls.

B.10 Southern Armyworm (Spodoptera Eridania)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, compounds 1-1, 1-2, 1-5, 1-6, and 1-9, resp., at 10 ppm showed over 75% mortality in comparison with untreated controls.

B.11 Green Soldier Stink Bug (Nezara viridula)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use. Soybean pods were placed in glass Petri dishes lined with moist filter paper and inoculated with ten late 3rd instar N. viridula. Using a hand atomizer, approximately 2 ml solution is sprayed into each Petri dish. Assay arenas were kept at about 25° C. Percent mortality was recorded after 5 days.

In this test, compounds 1-1, 1-2, 1-3, 1-4, 1-5, 1-6, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 2-1, and 3-1, resp., at 300 ppm showed over 75% mortality in comparison with untreated controls.

B.12 Neotropical Brown Stink Bug (Euschistus heros)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use.

Soybean pods were placed in microwavable plastic cups and inoculated with ten adult stage E. heros. Using a hand atomizer, approximately 1 ml solution is sprayed into each cup, insects and food present. A water source was provided (cotton wick with water). Each treatment was replicated 2-fold. Assay arenas were kept at about 25° C. Percent mortality was recorded after 5 days.

In this test, compounds 1-1, 1-2, 1-5, 1-6, 1-8, 1-9, 1-10, 1-12, 1-15, 1-16, 1-17, 1-18, 1-19, 1-21, 1-22, 1-23, 1-24, and 1-25, resp., at 500 ppm showed over 75% mortality in comparison with untreated controls.

B.13 Brown Marmorated Stink Bug (Halyomorpha halys)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use.

Row peanuts and soybean seeds were placed into microwavable plastic cups and inoculated with five adult stage H. halys. Using a hand atomizer, approximately 1 ml solution is sprayed into each cup, insects and food present. A water source was provided (cotton wick with water).

Each treatment is replicated 4-fold. Assay arenas are kept at about 25° C. Percent mortality was recorded after 5 days.

In this test, compounds 1-1, 1-2, 1-5, 1-6, 1-8, 1-9, 1-10, 1-11, 1-12, 1-13, 1-15, 1-16, 1-17, 1-18, 1-19, 1-20, 1-21, 1-22, 1-23, 1-24, and 1-25, resp., at 100 ppm showed over 75% mortality in comparison with untreated controls.

1. A glutarimide compound of formula I 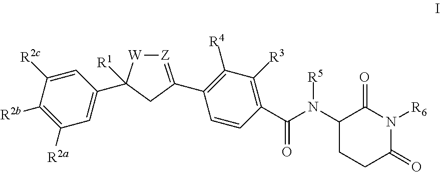
wherein
W—Z is —O—N═, —CH₂—N═, or —CH₂—CH═;
R¹ halomethyl;
R^{2a} halogen, halomethyl, or halomethoxy;
R^{2b}, R^{2c} are independently H, or as defined for Rea;
R³ is halogen, CN, NO2, C, C₂ alkyl, halomethyl, C₁-C₂-alkoxy, alkyl, C₁-C₂-haloalkoxy, or S(O)ₘ—C₁-C₂-haloalkyl;
R⁴ is H, or as defined for R³; or
R³ and R⁴ form together with the C-atoms they are bound to a 5-, or 6-membered saturated, partially, or fully unsaturated carbocyclic ring;
R⁵, R⁶ are independently H, CN, C, C₁-C₁₀ alkyl, C₃-C₈-cycloalkyl, C₂-C₁₀-alkenyl, C₃-C₈-cycloalkenyl, C₂-C₁₀-alkynyl, OR¹⁰, S(O)ₘR¹⁰, S(O)ₘN(R¹⁰)₂, N(R¹⁰)₂, which aliphatic groups are unsubstituted, partially or fully halogenated and/or substituted with one or more R^{a}; phenyl which is unsubstituted or substituted with one or more R^{A}; and 3- to 7-membered saturated, partially or fully unsaturated heterocycle comprising 1, 2 or 3 heteroatoms 0, N(O)ₙ or S(O)ₘ as ring members, which heterocycle is unsubstituted or substituted with one or more R^{A},
R¹⁰ is independently H, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, which groups are unsubstituted or substituted with one or more R^{a},
R^{a} is CN, N₃, NO₂, SCN, SF₅, Si(C₁-C₄-alkyl)₃, OR^{a1}, OSO₂R^{a1}, S(O)ₘR^{a1}, N(R^{a2})R^{a3}, C(═O)N(R^{a2})R^{a3}, C(═S)N(R^{a2})R^{a3}, C(═O)R^{a1}, C(═O)OR^{a1}, CH═NOR^{a1}, C₃-C₈-cycloalkyl, C₃-C₈-halocycloalkyl, which cyclic moieties may be substituted with R^{a4}; phenyl which is unsubstituted or substituted with one or more R^{A}; and 3- to 7-membered saturated, partially or fully unsaturated heterocycle comprising 1, 2 or 3 heteroatoms O, N(O)ₙ or S(O)ₘ as ring members, which heterocycle is unsubstituted or substituted with one or more R^{A},
m is 0, 1, or 2;
n is 0, or 1;
R^{a1} H, C₁-C₆-alkyl, C₁-C₆-haloalkyl, C₂-C₄-alkenyl, C₂-C₄-alkynyl, CH₂—CN, C₃-C₆-cycloalkyl, C₃-C₆-halocycloalkyl, C₃-C₆-cycloalkylmethyl, C₃-C₆-halocycloalkylmethyl, phenyl and hetaryl which aromatic rings are unsubstituted or partially or fully substituted with R^{A};
R^{a2} is H, or C₁-C₆-alkyl,
R^{a3} is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, or C₃-C₆-cycloalkyl, C₃-C₆-halocycloalkyl, C₃-C₆-cycloalkylmethyl, or C₃-C₆-halocycloalkylmethyl which rings are unsubstituted or substituted with a cyano;
R^{a4} is independently OH, CN, C₁-C₆-alkoxy, C₁-C₆-haloalkoxy, S(O)ₘ—C₁-C₆-alkyl, S(O)ₘ—C₁-C₆-haloalkyl, C(═O)N(R^{a2})R^{a3}, C₃-C₆-cycloalkyl, or C₃-C₆-halocycloalkyl which cycles are unsubstituted or substituted with one or more R^{a11}; or
phenyl, partially or fully unsaturated heterocycle which rings are unsubstituted or substituted with one or more R^{A};
R^{a11} is independently OH, cyano, C₁-C₂-alkyl, or C₁-C₂-haloalkyl;
R^{A} is independently selected from halogen, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, S(O)ₘ—C₁-C₄-alkyl, S(O)ₘ—C₁-C₄-haloalkyl, C₁-C₄-alkylcarbonyl, C₁-C₄-haloalkylcarbonyl, C(═O)N(R^{a2})R^{a3}; or
two R^{A} present on the same carbon atom of a saturated or partially saturated ring may form together ═O or ═S; or
two R^{A} present on the same S or SO ring member of a heterocyclic ring may together form a group ═N(C₁-C₆-alkyl), ═NO(C₁-C₆-alkyl), ═NN(H)(C₁-C₆-alkyl) or ═NN(C₁-C₆-alkyl)₂;
and the N-oxides, stereoisomers and agriculturally or veterinarily acceptable salts thereof.

2. The compound of formula I according to claim 1, which corresponds to formula I.a

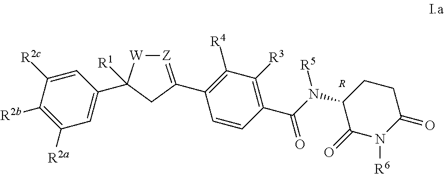

3. The compound of formula I according to claim 1, which corresponds to formula I.Aa

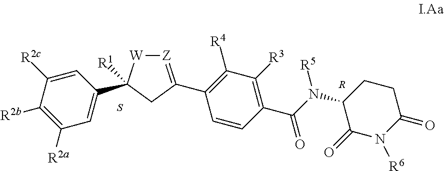

4. The compound of formula I according to claim 1, which corresponds to formula I.1

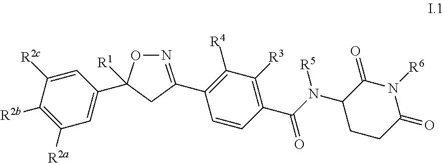

5. The compound of formula I according to claim 1, which corresponds to formula I.2

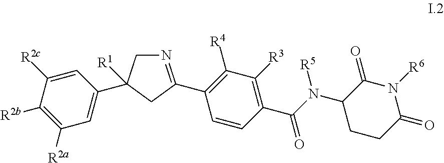

6. The compound of formula I according to claim 1, which corresponds to formula I.3

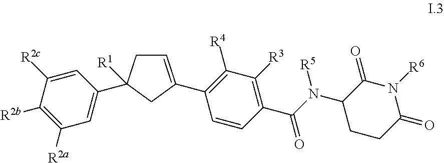

7. The compound of formula I according to claim 1, wherein R¹ is halomethyl.

8. The compound of formula I according to claim 1, wherein R³ is halogen, NO₂, CN, CH₃, fluoromethyl, CF₃, SCH₃, or OCH₃, and R⁴ is H.

9. The compound of formula I according to claim 1, wherein R⁵ is H, C₁-C₆-alkyl, C₃-C₆-cycloalkyl, C₂-C₆-alkenyl, or C₂-C₆-alkynyl.

10. The compound of formula I according to claim 1, wherein R⁶ is C₁-C₆-alkyl which is unsubstituted or substituted with phenyl or C(═O)—C₁-C₆-alkoxy; or R⁶ is C₁-C₄-alkyl, C₁-C₃-haloalkyl, C₃-C₄-cycloalkyl, C₁-C₃-alkoxy, C₁-C₃-haloalkoxy, C₁-C₄-alkylamino, and di-C₁-C₄-alkylamino.

11. The compound of formula I according to claim 1, wherein R¹ is halomethyl; R^{2a}, R^{2b}, R^{2c} are each selected from H, Cl, and F; R³ is Cl or CH₃; R⁴ is H, or R³ and R⁴ together form a C₃-carbon chain; R⁵ is H; and R⁶ is H, C₁-C₄-alkyl, C₁-C₂-haloalkyl, c-C₃H₅, C₁-C₂-alkoxy, or di-C₁-C₄-alkylamino.

12. A composition comprising at least one compound according to claim 1 and/or at least one agriculturally acceptable salt thereof, and at least one inert liquid and/or solid agriculturally acceptable carrier.

13. An agricultural composition for combating animal pests comprising at least one compound as defined in claim 1 and at least one inert liquid and/or solid acceptable carrier and, if desired optionally, at least one surfactant.

14. A method for combating or controlling invertebrate pests, comprising contacting said pest or its food supply, habitat, or breeding grounds with a pesticidally effective amount of at least one compound as defined in claim 1.

15. A method for protecting growing plants from attack or infestation by invertebrate pests, comprising contacting a plant, or soil or water in which the plant is growing, with a pesticidally effective amount of at least one compound as defined in claim 1.
